(19) United States
(12) Patent Application Publication
(10) Pub. No.: US 2017/0219518 A1
(43) Pub. Date: Aug. 3, 2017

(54) NON-INVASIVE ION RESPONSIVE URINE SENSOR

(71) Applicants: KOREA INSTITUTE OF SCIENCE AND TECHNOLOGY, Seoul (KR); THE ASAN FOUNDATION, Seoul (KR); UNIVERSITY OF ULSAN FOUNDATION FOR INDUSTRY COOPERATION, Ulsan (KR)

(72) Inventors: Kwan Hyi LEE, Seoul (KR); Min Hong JEUN, Seoul (KR); Sung Wook PARK, Seoul (KR); Seung Jae MYUNG, Seoul (KR); Choung Soo KIM, Seoul (KR); In Gab JEONG, Seoul (KR); Sang Hoon SONG, Seoul (KR)

(73) Assignees: KOREA INSTITUTE OF SCIENCE AND TECHNOLOGY, Seoul (KR); THE ASAN FOUNDATION, Seoul (KR); UNIVERSITY OF ULSAN FOUNDATION FOR INDUSTRY COOPERATION, Ulsan (KR)

(21) Appl. No.: 15/371,284

(22) Filed: Dec. 7, 2016

(30) Foreign Application Priority Data
Jan. 28, 2016 (KR) 10-2016-0010732

(51) Int. Cl. G01N 27/414; C12Q 1/68; G01N 33/493; G01N 33/543; G01N 33/574

(52) U.S. Cl. CPC G01N 27/4145; G01N 27/4146; G01N 33/54373; G01N 33/57434; G01N 33/57484; G01N 33/493; C12Q 1/6825; C12Q 1/682

(57) ABSTRACT

Provided is a semiconductor-based ion-responsive urine sensor (IRUS) capable of detecting an analyte in urine by a non-invasive method. When a urine sensor according to an aspect is used, it is possible to diagnose a patient accurately in a comfortable condition and to use the urine sensor for point-of-care (POC) diagnosis.

### CROSS-REFERENCE TO RELATED APPLICATION

This application claims the benefit of Korean Patent Application No. 10-2016-0010732, filed on Jan. 28, 2016, in the Korean Intellectual Property Office, the disclosure of which is incorporated herein in its entirety by reference.

### BACKGROUND

1. Field

One or more embodiments relate to a semiconductor-based ion-responsive urine sensor (IRUS) capable of detecting an analyte in urine by using a non-invasive method.

2. Description of the Related Art

Studies on the diagnosis of diseases using urine began in earnest in 1958 with the development of a urine dipstick that could detect glucose and protein. Since then, research on a system for diagnosing more diseases and a testing system using urine is underway. Techniques that can diagnose various diseases, including prostate cancer, using urine should minimize the patient's stress upon testing and diagnosis, and also enable more accurate diagnosis with the patient in a relaxed state. Repeated and persistent monitoring of diseases should also be possible for both children and older patients without causing mental and physical burdens.

Prostate cancer, which can be diagnosed through urine, is the fourth highest cancer in terms of incidence rate among all cancers, and is the second highest cancer in terms of incidence rate in men, accounting for about 15% of male cancers. In addition, prostate cancer has the fifth highest mortality rate among all cancers, with about 307,000 deaths in 2012. The technique for early diagnosis of prostate cancer that is currently in use is based on the detection of prostate-specific antigen (PSA) from a blood sample. Such diagnosis of prostate cancer is based on whether the PSA concentration exceeds 4 nanograms per millileter (ng/mL); however, prostate cancer is often diagnosed even with a concentration lower than the reference value. Thus, it is difficult to confirm a diagnosis of prostate cancer with a single diagnostic method. In addition, a biopsy is indispensable for suspected patients; however, through these tests, false-positive results are obtained with a total probability of ⅔. Furthermore, these methods are painful for patients, and due to misdiagnosis, the patients are not properly treated according to the tumor stage. Thus, these methods are a burden to the patients. Current diagnostic methods for prostate cancer are predominantly non-invasive, which can cause the patients to become uncooperative or cause them discomfort due to the repetitive sample collection and testing required, which in itself may also lead to a higher likelihood of a secondary condition such as an infection.

Thus, there is a need for a device/sensor and diagnostic technique that can diagnose and monitor diseases using urine in a non-invasive manner.

### SUMMARY

One or more embodiments include a sensor for urinalysis, wherein the sensor includes an electrochemical sensing unit for detecting an analyte in urine and a signal processor that includes an ion-sensitive field-effect transistor (ISFET) electrically connected to the sensing unit.

Additional aspects will be set forth in part in the description which follows and, in part, will be apparent from the description, or may be learned by practice of the presented embodiments.

According to one or more embodiments, provided is a sensor for urinalysis, the sensor including an electrochemical sensing unit for detecting an analyte in urine and a signal processor including an ion-sensitive field-effect transistor (ISFET) electrically connected to the sensing unit.

In an embodiment, the sensor may include an electrochemical sensing unit for detecting an analyte in urine and a signal processor including an ISFET for amplifying signals generated from the sensing unit, wherein the ISFET is electrically connected to the sensing unit, the sensing unit may be separable from the signal processor, and an electrode of the sensing unit may be electrically connected to an upper gate electrode of the ISFET.

In another embodiment, the sensor may further include a connecting portion for connecting the sensing unit to the signal processor. The connecting portion may be configured to be separable from the sensing unit and, for example, the connecting portion may have the form of a plug.

In still another embodiment, the sensor may further include a display unit for displaying results obtained by the sensor. The display unit may further include a display showing the results and a frame including at least one control interface (e.g., a power button, scroll wheel, or the like). The frame may include a slot for into which a sensor may be inserted. The frame may include a circuit, and thus, when the frame is provided with a sample, the frame may apply an electrical potential or current to an electrode of a sensor. A suitable circuit that may be used in the meter may be, for example, an appropriate voltage meter capable of measuring the potential across the electrode. A switch may also be provided which may be open when the electrical potential is measured or closed for measuring the current. The switch may be a mechanical switch (for example, a relay switch) or a solid-state switch. Such a circuit may be used in measuring a potential or current difference. As can be appreciated by those skilled in the art, other circuits, including simpler and more complex circuits, may be used to achieve application of a potential difference or a current difference or both.

The sensing unit may include a substrate; a working electrode and a reference electrode both on the substrate; immobilized analyte binding materials on the working electrode; and a test cell for accommodating the electrodes, the analyte binding materials, and an analyte. The sensing unit may be disposable. For example, the substrate may be formed of a material selected from the group consisting of silicon, glass, metal, plastic, and ceramic. In detail, the substrate may include a material selected from the group consisting of silicon, glass, polystyrene, polymethyl acrylate, polycarbonate, and ceramic. The electrode may include, for example, silver, silver epoxy, palladium, copper, gold, platinum, silver/silver chloride, silver/silver ion, or mercury/mercuric oxide. The sensing unit may also include an insulating electrode on the substrate or on the working electrode. The insulating electrode may include a natural or artificially formed oxide film. Examples of the oxide film include SiₓO_{y}, HₓfO_{y}, AlₓO_{y}, TaₓO_{y}, and TiₓO_{y} (wherein x and y are each an integer from 1 to 5). The oxide film may be formed by a known method. For example, an oxide may be deposited on a substrate by liquid phase deposition, evaporation, or sputtering.

The terms “analyte binding materials” and “analyte binding reagents” as used herein are used interchangeably and refer to materials that may functionalize a sensing unit or analyte-specific binding materials. The analyte binding materials may include deoxyribonucleic acids (DNA), ribonucleic acids (RNA), nucleotides, nucleosides, proteins, polypeptides, peptides, amino acids, carbohydrates, enzymes, antibodies, antigens, receptors, substrates, ligands, membranes, or a combination thereof. For example, the analyte binding materials may be antibodies that specifically bind to prostate-specific antigen (PSA), Annexin A3 (ANX A3), or prostate-specific membrane antigen (PSMA), which are prostate cancer markers. The analyte binding materials may also include a redox enzyme. The redox enzyme may refer to an enzyme that oxidizes or reduces a substrate, and may include, for example, an oxidase, a peroxidase, a reductase, a catalase, or a dehydrogenase. The redox enzyme may include, for example, a glucose oxidase, a lactate oxidase, a cholesterol oxidase, a glutamate oxidase, a horseradish peroxidase (HRP), an alcohol oxidase, a glucose oxidase (GOx), a glucose dehydrogenase (GDH), a cholesterol esterase, an ascorbic acid oxidase, an alcohol dehydrogenase, a laccase, a tyrosinase, a galactose oxidase, or a bilirubin oxidase. The analyte binding materials may be immobilized on a substrate, a working electrode, or an insulating electrode, and the term “immobilized” as used herein may refer to a chemical or physical bond between analyte binding materials and a substrate. In addition, an immobilization compound may be immobilized on the substrate or the electrode. The immobilization compound may refer to a material capable of binding to an analyte or a linker for immobilizing an analyte binding material on a surface of a substrate or an electrode. The immobilization compound may be biotin, avidin, streptavidin, carbohydrate, poly L-lysine, a hydroxy group, a thiol group, an amine group, an alcohol group, a carboxyl group, an amino group, a sulfur group, an aldehyde group, a carbonyl group, a succinimide group, a maleimide group, an epoxy group, an isothiocyanate group, or a combination thereof.

The term “analyte” as used herein may refer to a material of interest that may be present in a sample. An analyte that may be detected may be that which specifically binds with at least one analyte binding material, and which may participate in a sandwich, competitive, or substitutive assay configuration. Examples of the analyte include an antigen such as a peptide (e.g., a hormone), a heptene, a protein (e.g., an enzyme), a carbohydrate, a drug, a pesticide, a microorganism, an antibody, and a nucleic acid that may participate in a sequence-specific hybridization reaction with a complementary sequence. For example, the analyte may be PSA, Annexin A3 , or PSMA, which are prostate cancer markers.

When the sensing unit is provided with a sample through a test cell for accommodating the electrodes, the analyte binding materials, and an analyte, an analyte present in the sample may bind to the analyte binding materials, thus causing a chemical potential gradient in the test cell. The term “chemical potential gradient” as used herein may refer to a concentration gradient of an active species. In the case that such a gradient is present between two electrodes, when a circuit is opened, a potential difference may be detected, and when the circuit is closed, a current may flow until the gradient disappears. The chemical potential gradient may refer to any potential gradient arising from a potential difference or application of a current flow between the two electrodes. The test cell may be prepared using polydimethylsiloxane (PDMS), polyethersulfone (PES), poly(3,4-ethylenedioxythiophene), poly(styrenesulfonate), polyimide, polyurethane,polyester, perfluoropolyether (PFPE), polycarbonate, or a combination of the foregoing polymers.

The ISFET may include a lower gate electrode; a lower insulating layer on the lower gate electrode; a source and a drain on the lower insulating layer and separated from each other; a channel layer on the lower insulating layer and between the source and the drain; an upper insulating layer on the source, the drain, and the channel layer; and an upper gate electrode on the upper insulating layer.

Due to super capacitive coupling generated in a dual-gate ISFET including the channel layer, a small surface potential voltage difference that occurs in the sensing unit may significantly amplify a threshold voltage variation of a lower field-effect transistor. In this regard, an amplification factor may be determined according to a thickness of the lower insulating layer, a thickness of the channel layer, a thickness of upper insulating layer. As the thickness of the lower insulating layer increases, and as the thickness of the upper insulating layer and the thickness of the channel layer decreases, the amplification factor may become larger.

The channel layer may be an ultra-thin film layer having a thickness, for example, of 10 nanometers (nm) or less, 9 nm or less, 8 nm or less, 7 nm or less, 6 nm or less, 5 nm or less, or 4 nm or less.

When the thickness of the channel layer is within any of these ranges, super capacitive coupling, which may control under all conditions up to the upper interface, may occur due to a strong electric field of the lower gate electrode induced at the ultra thin film. As a result, electrons and holes induced at an upper gate interface may be controlled, and current leakage may be blocked. In addition, by permitting a stable amplification factor, a linear response, hysteresis, and a drift phenomenon depending on a surface potential may improve, and the electrostatic coupling of the upper and lower gates may be sustained. Further, when the thickness of the channel layer is within any of the above ranges, a transistor including the ultra-thin channel layer may, as compared with a conventional transistor, have increased ion-sensing ability while permitting a larger amplification factor. Further, when the thickness of the channel layer is within any of the above ranges, a transistor including the ultra-thin channel layer may, as compared with a conventional transistor, have improved stability. The varying amplification factor seen in a thick channel layer may cause the deterioration of a device due to ion damage, by combination with the current leakage induced at an upper interface. On the other hand, a transistor according to an embodiment, in which the current leakage is controlled while permitting a constant amplification factor, may minimize an effect of ion damage. In addition, when the lower insulating layer is excessively thick in a conventional transistor, a lower electric field may not fully control a channel region, and thus the electrostatic coupling of the upper and lower gates may be weakened. However, a transistor including an ultra-thin channel layer according to an embodiment may achieve a large amplification factor while maintaining the electrostatic coupling. The capacitive coupling of the upper and lower gates occurs when the upper channel interface is completely depleted. In a conventional transistor, amplification may not occur because an electric field of a lower gate cannot control an upper channel.

The channel layer may include any one selected from the group consisting of an oxide semiconductor, an organic semiconductor, polycrystalline silicon, and monocrystalline silicon. When the channel layer includes any one selected from the group consisting of an oxide semiconductor, an organic semiconductor, polycrystalline silicon, and monocrystalline silicon, capacitive coupling of upper and lower gates may occur, a highly sensitive sensor may be manufactured, and a transparent and flexible sensor may be provided. A width or length of the channel layer is not limited, and capacitive coupling may be utilized by using upper and lower gate electrodes in a dual-gate structure.

Also, in the sensor, a thickness of an equivalent oxide layer of the upper insulating layer may be thinner a the thickness of an equivalent oxide layer of the lower insulating layer. For example, the thickness of the upper insulating layer may be about 25 nm or less, and the thickness of the lower insulating layer may be about 50 nm or greater. When the thickness of an equivalent oxide layer of the upper insulating layer is thinner than the thickness of an equivalent oxide layer of the lower insulating layer, amplification of signal sensitivity may occur.

The upper insulating layer and the lower insulating layer may include a natural or artificially formed oxide film. Examples of the oxide film include SiₓO_{y}, HₓfO_{y}, AlₓO_{y}, TaₓO_{y}, and TiₓO_{y} (wherein x and y may each be an integer from 1 to 5). The oxide layer may have a single, double, or triple-layered structure. Thus, by increasing the thickness and decreasing the thickness of an equivalent oxide layer of the upper insulating layer, the sensitivity of the sensor may be amplified, and deterioration thereof due to the leakage current may be prevented.

A dual-gate ISFET according to an embodiment may include both an upper field transistor including an upper insulating layer and a lower field transistor including a lower insulating layer in one device. Depending on respective modes of operation, each gate of the dual-gate ISFET may independently be operated as an upper gate or a lower gate. When upper and lower gates of a device are used simultaneously, the capacitive coupling may be observed due to the structural specificity of a dual-gate structure, and thus, correlation between upper and lower field transistors may be established. In a dual operation mode, a lower gate may be used as a main gate. Thus, a transistor according to an embodiment may be operated in a dual-gate mode.

In another embodiment, the sensing unit may further include a probe coupled to analyte binding materials via an analyte in a sample and having a negative charge or a positive charge. Signals of the analyte may be amplified by capacitive coupling of the probe to electrons in the channel layer of the transistor.

The probe may include metal nanoparticles. The metal nanoparticles may be, for example, gold nanoparticles, which may additionally supply charges. The probe may also include a quantum dot. When a quantum dot is used, the quantum dot may additionally supply charges as gold nanoparticles and also perform bioimaging at the same time. The probe may also include ferritin. The combined structure of ferritin and metal nanoparticles may provide larger signals by providing more charges than the single-metal nanoparticles.

In another embodiment, the sensor may include a plurality of sensing units for detecting an analyte and a plurality of ISFETS.

The sensor may include the plurality of sensing units and the plurality of ISFETs, wherein the plurality of sensing units may be electrically connected to the plurality of ISFETs, respectively. In the plurality of ISFETS, a plurality of sources may commonly be grounded, a plurality of upper gate electrodes may commonly be grounded, and a common voltage may be applied to a plurality of lower gate electrodes. For example, sources of a first transistor and a second transistor, and reference electrodes of the first sensing unit and the second sensing unit may commonly be grounded. For example, a common voltage may be applied to lower gate electrodes of the first transistor and the second transistor. In addition, a plurality of drains in the plurality of transistors may have a parallel structure. For example, drains of the first transistor and the second transistor may have a parallel structure. The plurality of sensing units may each independently include different immobilized analyte binding materials. For example, an antibody against PSA may be immobilized on the first sensing unit, and an antibody against PSMA may be immobilized on the second sensing unit. The plurality of transistors may sense the same or different analyte signals from the plurality of sensing units, amplify the signals, and output the signals through a semiconductor parameter analyzer.

In another embodiment, the signal processor may further include a calculation module electrically connected to the ISFET, for the calculation module determining an amount of the analyte in urine from a potential difference measured from the ISFET. The calculation module may be for the determination of an analyte. The term “determination of an analyte” as used herein may refer to a qualitative, semi-quantitative, or quantitative process for evaluating a sample. In a qualitative evaluation, the result indicates whether an analyte is detected in a sample. In a semi-quantitative evaluation, the result indicates whether an analyte is present above a predefined threshold value. In a quantitative evaluation, the result is a numerical indication of the amount of an analyte present therein. In order to convert measured values, a look-up table that converts a specific value of a current or a potential into a value of an analyte depending on a correction value for a specific device structure and an analyte may be used. The calculation module may determine an amount of an analyte by measuring a potential difference according to a known concentration of an analyte. For example, the calculation module may determine the amount of a prostate cancer marker in urine according to a graph shown in FIG. 7A, 7B, or 7C.

In still another embodiment, the sensor may include a communicator, which may allow the sensor to be the capable of transmitting/receiving information to/from an external server or a terminal unit. The communicator may employ a wired or wireless communicator. Therefore, wired communication via a cable connection may be used, and wireless communication, including via a 4G, LTE, UWB, WiFi, WCDMA, USN, or IrDA module, as well as a Bluetooth module or a Zigbee module, may be used.

The terminal unit may include a communication device such as a computer, a notebook computer, a smartphone, a general mobile phone, a personal digital assistant (PDA), and a measuring instrument or a control device having a separate communication function. The terminal unit may include a central processing unit and may be operating system (OS)-based, and may be capable of running software such as a computer program and an application program. Therefore, an application program, which is for reading, analyzing, and processing measurement data of an analyte in urine provided by the sensor, may be mounted to the terminal unit, thus enabling the terminal unit to read, analyze, and process the measurement data of the analyte in urine. The terminal unit may also display the measurement data of the analyte in urine or the read, analyzed, and processed measurement data of the analyte in urine. Also, the terminal unit may be connected to or linked with a control unit of a sensor, and thus the terminal unit may operate and control the sensor.

### BRIEF DESCRIPTION OF THE DRAWINGS

These and/or other aspects will become apparent and more readily appreciated from the following description of the embodiments, taken in conjunction with the accompanying drawings in which:

FIG. 1 is a schematic diagram illustrating a sensor according to an embodiment;

FIG. 2 is a cross-sectional diagram illustrating a sensing unit of a sensor according to an embodiment;

FIG. 3 is a cross-sectional diagram illustrating a dual-gate ion-sensitive field-effect transistor (ISFET) of a sensor according to an embodiment;

FIG. 4 is a schematic diagram illustrating a sensor using a probe, according to an embodiment;

FIG. 5 is a diagram illustrating a multiplexing detection system of a sensor according to an embodiment;

FIG. 6 is a graph of reference voltage (volts, V) versus time (minutes), illustrating the result of an evaluation of stability of a sensor according to an embodiment;

FIG. 7A is a graph of threshold voltage difference (ΔVth, V) versus analyte molar concentration (molar, M), illustrating the result of measurements of a potential difference according to a known concentration of prostate-specific antigen (PSA), the measurements obtained by a sensor according to an embodiment;

FIG. 7B is a graph of ΔVth (V) versus analyte molar concentration (M), illustrating the result of measurements of a potential difference according to a known concentration of Annexin A3 (ANX A3), the measurements obtained by a sensor according to an embodiment;

FIG. 7C is a graph of ΔVth (V) versus analyte molar concentration (M), illustrating the result of measurements of a potential difference according to a known concentration of prostate-specific membrane antigen (PSMA), the measurements obtained by a sensor according to an embodiment;

FIG. 8 is a series of graphs of ΔVth (V) versus analyte molar concentration (M), illustrating the results of measurements of PSA, ANX A3, and PSMA in patients' urine, the measurements obtained by a sensor according to an embodiment;

FIG. 9 is a histogram of sensing sigma (voltage difference, ΔV) according to methods of collecting urine from a patient, illustrating the result of measurements of ANX A3, the measurements obtained by a sensor according to an embodiment; and

FIG. 10 shows tables illustrating the results of measurements of PSA, ANX A3, and PSMA in patients' urine, the measurements obtained by a sensor according to an embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description.

Most of the terms used herein are general terms that have been widely used in the technical art to which the present invention pertains. However, some of the terms used herein may be created reflecting intentions of technicians in this art, precedents, or new technologies. Also, some of the terms used herein may be arbitrarily chosen by the present applicant. In this case, these terms are defined in detail below. Accordingly, the specific terms used herein should be understood based on the unique meanings thereof and the whole context of the present invention.

Throughout the specification, it will be understood that when an element is referred to as being “connected” to another element, it may be “directly connected” to the other element or “electrically connected” to the other element with intervening elements therebetween. The terms, such as “including” or “having”, are intended to indicate the existence of the elements disclosed in the specification, and are not intended to preclude the possibility that one or more other elements may exist or may be added. Also, the terms “unit” or “module” as used herein should be understood as a unit that processes at least one function or operation and that may be embodied in a hardware manner, a software manner, or a combination of the hardware manner and the software manner.

The terms “configured” or “included” as used herein should not be construed to include all of various elements or steps described in the specification, and should be construed to not include some of the various elements or steps or to further include additional elements or steps.

The following description of the embodiments should not be construed as limiting the scope of the present invention, and modifications that those of skilled in the art can readily infer from the present invention should be construed as being within the scope of the present invention. Hereinafter, exemplary embodiments for descriptive sense only will be described in detail with reference to the accompanying drawings.

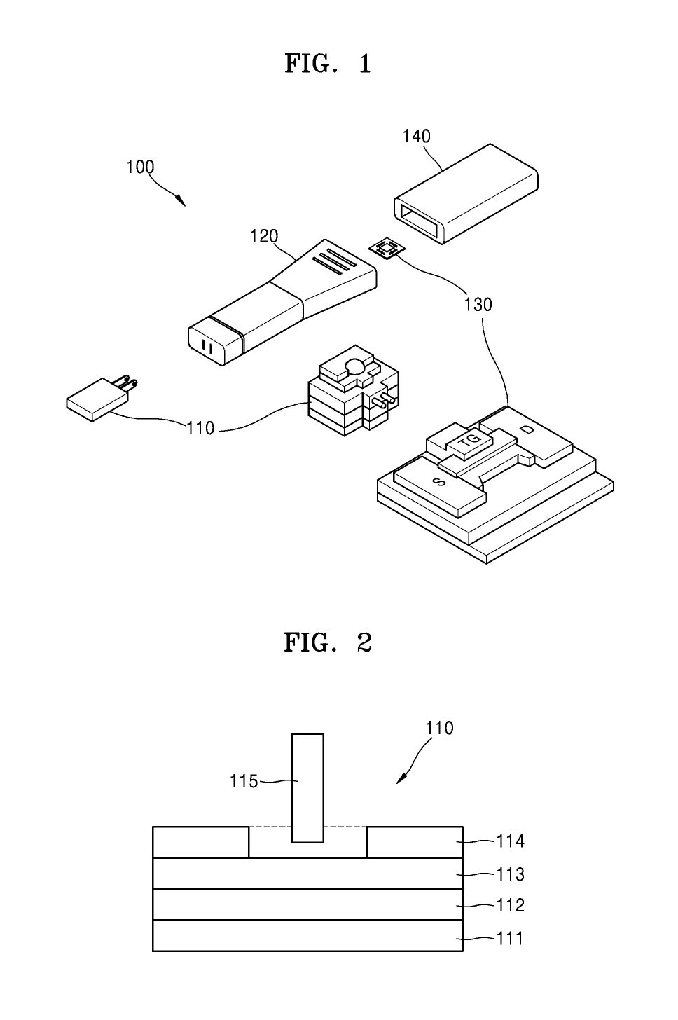

FIG. 1 is a schematic diagram illustrating a sensor according to an embodiment. Referring to FIG. 1, a sensor 100 according to an embodiment may include a sensing unit 110 for detecting an analyte in urine and an ion-sensitive field-effect transistor (ISFET) 130 electrically connected to the sensing unit 110. In an embodiment, the sensor 100 may include the electrochemical sensing unit 110 for detecting an analyte in urine and a signal processor 130 for amplifying signals generated from the sensing unit 110, wherein the signal processor 130 may include the ISFET 130 electrically connected to the sensing unit 110, the sensing unit 110 may be separable from the signal processor 130, and an electrode of the sensing unit 110 may be electrically connected to an upper gate electrode of the ISFET 130. In another embodiment, the sensor 100 may further include a connecting portion 120 for connecting the sensing unit 110 to the signal processor 130. The connecting portion 120 may be configured to be separable from the sensing unit 110 and, for example, the connecting portion 120 have the form of a plug. In still another embodiment, the sensor 100 may further include a display unit 140 for displaying results obtained by the sensor 100. The display unit 140 may further include a display showing the results and a frame including at least one control interface (e.g., a power button, scroll wheel, or the like). The frame may include a slot for into which a sensor may be inserted. The frame may include a circuit, and thus, when the frame is provided with a sample, the frame may apply an electrical potential or current to an electrode of the sensor 100. A suitable circuit that may be used in the meter may be, for example, an appropriate voltage meter capable of measuring the potential across the electrode. A switch may also be provided which may be open when the electrical potential is measured or closed for measuring the current.

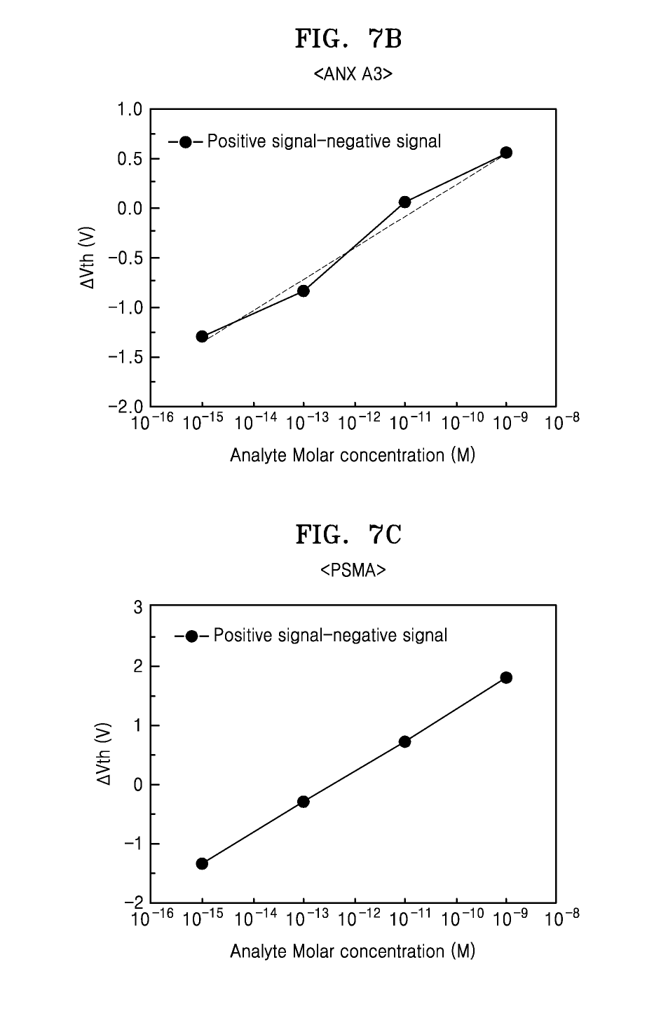

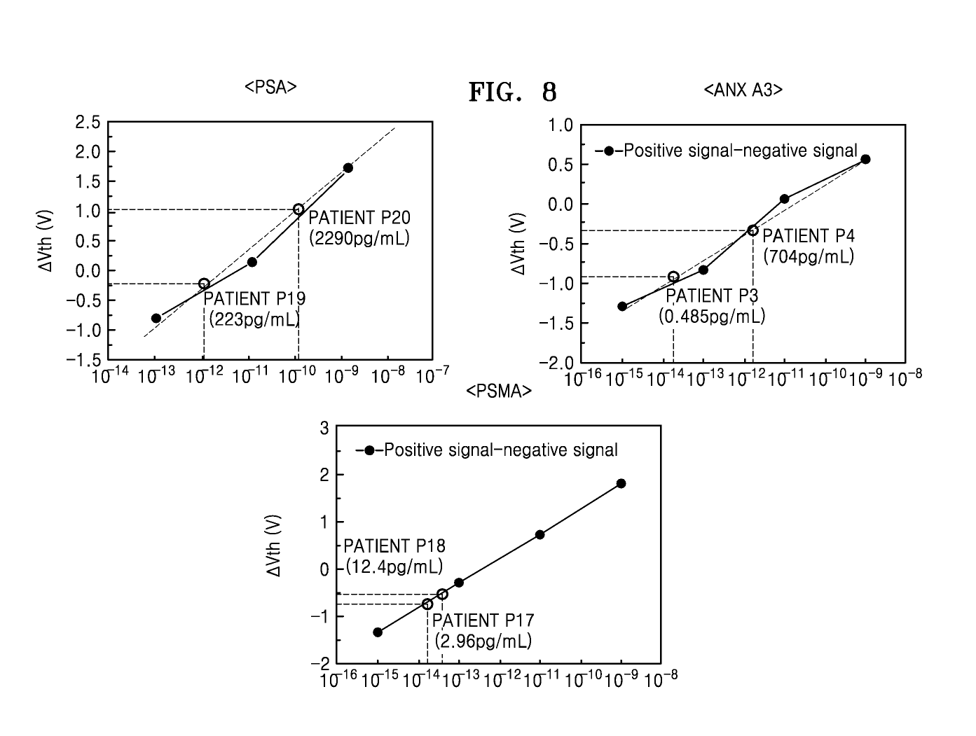

In another embodiment, the signal processor 130 may further include a calculation module (not shown), which may be electrically connected to the ISFET 130, for determining the amount of the analyte in urine from a potential difference measured by the ISFET 130. The calculation module may determine an analyte. The calculation module may determine the analyte by measuring a potential difference according to a known concentration of the analyte. For example, the calculation module may determine the amount of a prostate cancer marker in urine according to a graph shown in FIG. 7A, 7B, or 7C. In still another embodiment, the sensor 100 may include a communicator (not shown) which may provide the sensor 100 with the capability to transmit/receive information to/from an external server or a terminal unit. The communicator may employ a wired or wireless communicator.

FIG. 2 is a cross-sectional diagram illustrating a sensing unit of a sensor according to an embodiment. Referring to FIG. 2, the sensing unit 110 may include a substrate 111; a working electrode 112 and a reference electrode 115 on the substrate; immobilized analyte binding materials on the working electrode 112; and a test cell 114 for accommodating the electrodes 112 and 115, the analyte binding materials, and an analyte. The sensing unit 110 may be disposable. For example, the substrate may be include a material selected from the group consisting of silicon, glass, metal, plastic, and ceramic. The electrodes 112 and 115 may include, for example, silver, silver epoxy, palladium, copper, gold, platinum, silver/silver chloride, silver/silver ion, or mercury/mercuric oxide. The sensing unit 110 may also include an insulating electrode 113 provided on the substrate 111 or on the working electrode 112. The insulating electrode 113 may include a natural or artificially formed oxide film. Examples of the oxide film include SiₓO_{y}, HₓfO_{y}, AlₓO_{y}, TaₓO_{y}, and TiₓO_{y} (wherein x and y may each be an integer from 1 to 5). The oxide film may be formed by a known method. For example, an oxide may be deposited on a substrate by liquid phase deposition, evaporation, or sputtering. The analyte binding materials may include deoxyribonucleic acids (DNA), ribonucleic acids (RNA), nucleotides, nucleosides, proteins, polypeptides, peptides, amino acids, carbohydrates, enzymes, antibodies, antigens, receptors, substrates, ligands, membranes, or a combination thereof. For example, the analyte binding materials may be antibodies that specifically bind to prostate-specific antigen (PSA), Annexin A3 (ANX A3), or prostate-specific membrane antigen (PSMA), which are prostate cancer markers. Examples of the analyte may include an antigen such as a peptide (e.g., a hormone), a heptene, a protein (e.g., an enzyme), a carbohydrate, a protein, a drug, a pesticide, a microorganism, an antibody, and a nucleic acid that may participate in a sequence-specific hybridization reaction with a complementary sequence. For example, the analyte may include PSA, Annexin A3, or PSMA, which are prostate cancer markers. When the sensing unit 110 is provided with a sample through the test cell 114 accommodating the electrodes 112 and 115, the analyte binding materials, and an analyte, an analyte present in the sample may bind to the analyte binding materials to thereby cause a chemical potential gradient in the test cell 114.

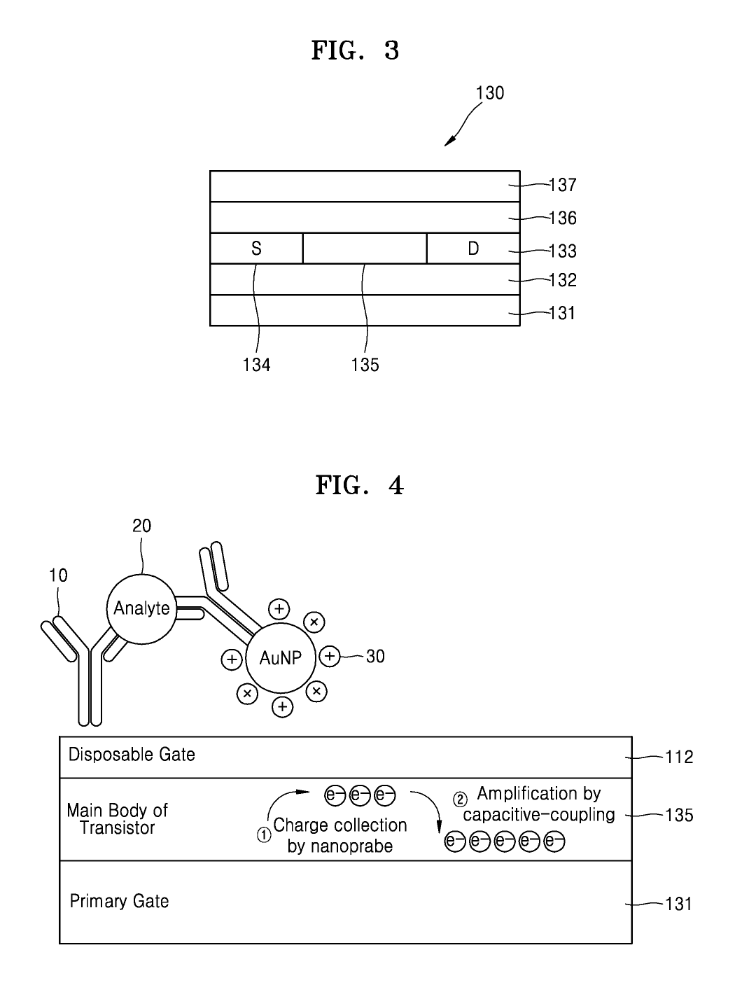

FIG. 3 is a cross-sectional diagram illustrating a dual-gate ISFET of a sensor according to an embodiment. Referring to FIG. 3, regarding the dual-gate ISFET, the ISFET 130 may include a lower gate electrode 131; a lower insulating layer 132 on the lower gate electrode 131; a source 134 and a drain 133 on the lower insulating layer 132 and separated from each other; a channel layer 135 on the lower insulating layer 132 and between the source 134 and the drain 133; an upper insulating layer 136 on the source 134, the drain 133, and the channel layer 135; and an upper gate electrode 137 on the upper insulating layer 136. Due to super capacitive coupling generated in the dual-gate ISFET 130 including the channel layer 135, a small surface potential voltage difference that occurs in the sensing unit may significantly amplify a threshold voltage variation of a lower field-effect transistor. In this regard, an amplification factor may be determined according to a thickness of the lower insulating layer 132, a thickness of the channel layer 135, a thickness of the upper insulating layer 136. As the thickness of the lower insulating layer 132 increases, and as the thickness of the upper insulating layer 136 and the thickness of the channel layer 135 decreases, the amplification factor may become larger. The channel layer 135 may be an ultra-thin film layer having a thickness, for example, of 10 nanometers (nm) or less, 9 nm or less, 8 nm or less, 7 nm or less, 6 nm or less, 5 nm or less, or 4 nm or less. The channel layer 135 may include any one selected from the group consisting of an oxide semiconductor, an organic semiconductor, polycrystalline silicon, and monocrystalline silicon. Also, in the sensor, a thickness of an equivalent oxide layer of the upper insulating layer 136 may be less than a thickness of an equivalent oxide layer of the lower insulating layer 132. For example, the thickness of an equivalent oxide layer of the upper insulating layer 136 may be about 25 nm or less, and the thickness of an equivalent oxide layer of the lower insulating layer 132 may be about 50 nm or greater. When the thickness of an equivalent oxide layer of the upper insulating layer 136 is less than the thickness of an equivalent oxide layer of the lower insulating layer 132, amplification of signal sensitivity may occur. A dual-gate ISFET 130 according to an embodiment may include both an upper field-effect transistor including an upper insulating layer 136 and a lower field-effect transistor including a lower insulating layer 132 in one device. Depending on respective modes of operation, each gate of the dual-gate ISFET may independently be operated as an upper gate or a lower gate. When upper and lower gates of a device are used simultaneously, capacitive coupling may be observed due to the structural specificity of the dual-gate structure, and thus, the correlation between upper and lower field-effect transistors may be established. In a dual operation mode, a lower gate may be used as a main gate. Thus, a transistor according to an embodiment may be operated in a dual-gate mode.

FIG. 4 is a schematic diagram illustrating a sensor using a probe according to an embodiment. Referring to FIG. 4, the sensing unit may further include a probe 30 coupled to analyte binding materials 10 via an analyte in a sample and having a negative charge or a positive charge. Signals of the analyte 20 may be amplified by capacitive coupling of the probe 30 to electrons in the channel layer 135 of the transistor.

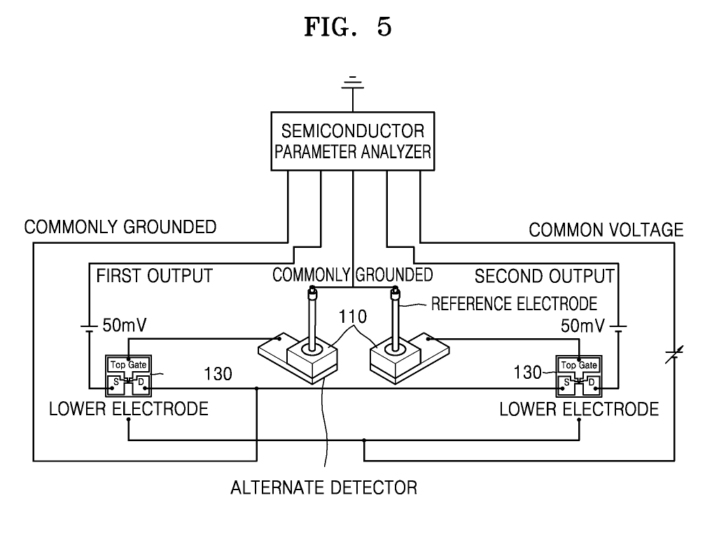

FIG. 5 is a diagram illustrating a multiplexing detection system of a sensor according to an embodiment. Referring to FIG. 5, regarding a multiplexing detection system of a sensor, the sensor may include a plurality of transistors 130 and a plurality of sensing units 110 for detecting an analyte. The sensor may include the plurality of sensing units 110 and the plurality of ISFETs 130, wherein the plurality of sensing units 110 may respectively be electrically connected to the plurality of ISFETs 130. In the plurality of transistors 130, a plurality of sources may commonly be grounded, a plurality of upper-gate electrodes may commonly be grounded, and a common voltage may be applied to a plurality of lower-gate electrodes. In addition, a plurality of drains in the plurality of transistors 130 may have a parallel structure. The plurality of sensing units 110 may each independently include different immobilized analyte binding materials. The plurality of transistors 130 may sense the same or different analyte signals from the plurality of sensing units 110, amplify the signals, and output the signals through a semiconductor parameter analyzer.

Example Manufacture of Sensor and Analysis of Characteristics

(1) Manufacture of Sensor for Urinalysis

(1.1) Manufacture of Dual-Gate ISFET

A silicon-on-insulator (SOI) substrate having resistivity of about 10 ohm-centimeter (Ω-cm) to 20 Ωcm was prepared, a thickness of silicon as a lower-gate electrode was about 107 nm, and a thickness of a buried SiO₂ oxide film as a lower insulating film was about 224 nm. After performing standard RCA cleaning, the upper silicon was etched with about 2.38 percent by weight (wt %) of a tetramethylammonium hydroxide (TMAH) solution to form an ultra-thin film, and a channel region was formed by photolithography. In this case, a length, a width, and a thickness of the channel were respectively about 20 micrometers (μm), 20 μm, and 4.3 nm. Subsequently, n-type polycrystalline silicon was deposited using a chemical vapor deposition (CVD) apparatus to form a source and a drain. Then, an upper insulating layer was formed by oxidizing silicon dioxide of a thickness of about 23 nm on the source and the drain. Next, to form an upper gate electrode, an Al thin layer having a thickness of about 150 nm was deposited on the upper insulating layer using an electron beam (e-beam) evaporator. Next, to remove defects and improve an interfacial state therebetween, heat treatment was performed at a temperature of about 450° C. in a gas atmosphere including N₂ and H₂, thereby completing the manufacture of a dual-gate ISFET.

(1.2) Manufacture of an Electrochemical Sensing Unit

In order to prepare an electrochemical sensing unit, SiO₂ having a thickness of about 300 nm was grown to form p-type silicon which was used as a substrate. After standard RCA cleaning was performed thereon, a working electrode of titanium (Ti) was deposited on the substrate at a thickness of about 100 nm using an e-beam evaporator to measure the electrical potential difference. Next, as an insulating electrode, a SnO₂ film was deposited on the Ti layer to a thickness of about 45 nm using an RF sputtering method with power of about 50 watts (W). Thereafter, a sputtering process was performed under an Ar gas atmosphere with a flow rate of about 20 standard cubic centimeters (sccm) and a pressure of about 3 milliTorr (mTorr). Next, a test cell for accommodating a sample was prepared from polydimethylsiloxane (PDMS) and attached onto the insulating electrode to prepare a sensing unit. In addition, a silver/silver chloride electrode was used as a reference electrode.

(1.3) Manufacture of Sensor

A sensor for urinalysis was prepared by connecting the upper gate electrode of the transistor prepared in (1.1) to the working electrode of the sensing unit prepared in (1.2) by a plug-in method.

(2) Analysis of Characteristics of Sensor

(2.1) Evaluation of Sensor Stability

In order to evaluate stability of the sensor prepared in (1.3), a signal was measured while alternately applying human urine and a pH 10 solution.

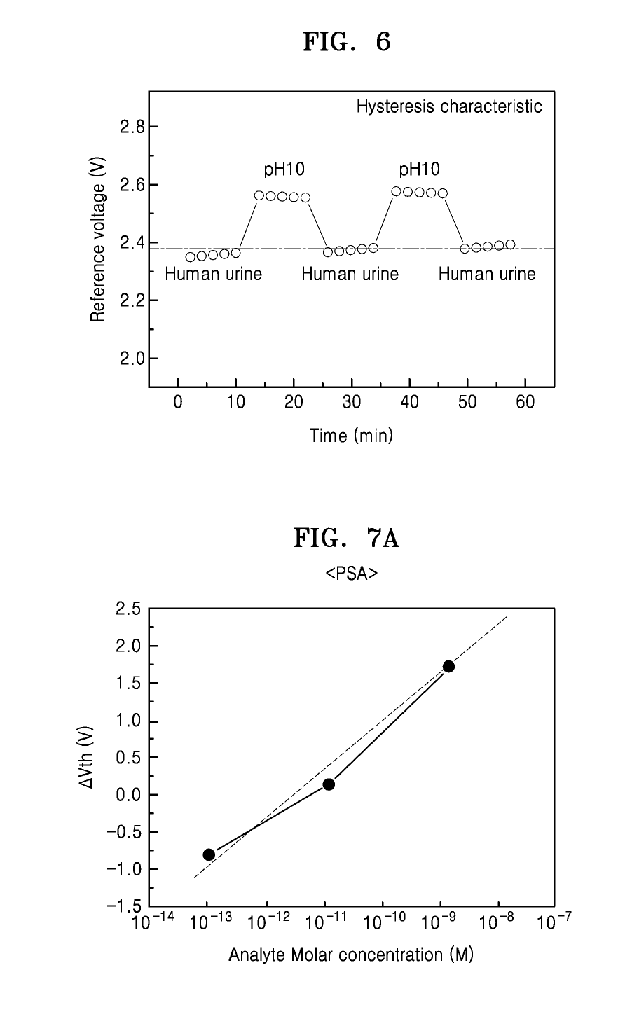

Specifically, human urine was obtained from Asan Medical Center, Seoul, Korea. The pH 10 solution was prepared by adding NaOH to distilled water while using a pH meter. First, a human urine sample was injected into the sensor and reacted for 10 minutes, and then the human urine sample was removed therefrom. Subsequently, the pH 10 solution was injected thereto for 10 minutes of reaction, and after the pH 10 solution was removed therefrom, the human urine sample was injected again thereto for 10 minutes of reaction. This process was repeated so as to analyze how the signals of the sensor varied. The evaluation results are shown in FIG. 6.

FIG. 6 is a graph illustrating the result of an evaluation of stability of the sensor according to an embodiment.

As shown in FIG. 6, it can be seen that even though different solutions were alternately injected into the sensor according to an embodiment, the reference voltage measured was consistent for each solution. Accordingly, the sensor according to an embodiment was found to measure electrical signals stably.

(2.2) Detection of Prostate Cancer Markers PSA, ANX A3, PSMA

To detect prostate cancer markers PSA, ANX A3, and PSMA (i.e., analyte), respective analyte binding materials such as antibodies for each analyte of PSA (available from Biorbyt), ANX A3 (available from Abnova), and PSMA (available from Abcam) were immobilized. An EDC/Sulfo-NHS reaction was performed to form —COOH groups on a surface of the insulating electrode of the sensor, and thus, —NH₂ groups of the antibodies were bound thereto such that the antibodies were immobilized. Urine samples from healthy adults and patients clinically diagnosed with prostate cancer were obtained from Asan Medical Center, Seoul, Korea.

First, the minimum signal of the sensor was normalized using a phosphate buffered saline (PBS) solution. In an experimental group, to stabilize the initial electrical signal, the PBS solution was injected into the sensing unit having an immobilized antibody against a specific prostate cancer marker. Thereafter, a urine sample of an actual clinical patient was injected into the sensing unit and reacted for 20 minutes and, subsequently, measurement was performed under a urine condition. In the control group, the signal of the urine itself was measured, which was performed to remove the background noise from the experimental group results. After the initial signal was stabilized by injecting the PBS solution into the sensing unit in which an antibody against a specific prostate cancer marker was not immobilized, the same urine used in the experimental group was injected and reacted for 20 minutes. Thereafter, measurement was performed. The final result was obtained by removing the control group signal from the experimental group signal.

To obtain a reference result that is used to quantitatively analyze an amount of an analyte in a sample, artificial urine containing a known concentration of the analyte was used (the known concentration: 1.5 L of distilled water (D.I), 36.4 g of urea, 15.0 g of sodium chloride, 9.0 g of potassium chloride, 9.6 g of sodium phosphate, 4.0 g of creatinine, and 100 mg of albumin). In detail, to measure a potential difference, artificial urine containing PSA, ANEX A3, or PSMA in a range of 10⁻¹⁵ g/mL to 10⁻⁹ g/mL was injected to a sensor having an immobilized antibody corresponding to the selected marker. The measurement results are shown in FIGS. 7A to 7C.

Each of FIGS. 7A to 7C show a graph illustrating the results of measurements of potential differences according to a known concentration of PSA, ANX A3, and PSMA, respectively, the measurements obtained by using a sensor according to an embodiment. Using the results of FIGS. 7A to 7C as a reference, the amounts of PSA, ANX A3, and PMSA may be quantitatively measured in urine of an actual clinical patient.

Next, an analyte, that is, PSA, ANX A3, or PMSA, was quantitatively detected in the urine of actual prostate cancer patients. Using the results of FIGS. 7A to 7C as a reference, each analyte (antigen) was quantitatively detected for two patients. The results are shown in FIG. 8.

FIG. 8 shows graphs illustrating the results of measurements of PSA, ANX A3, and PSMA in patients' urine, the measurements obtained by using a sensor according to an embodiment.

As shown in FIG. 8, the prostate cancer markers PSA, ANX A3, and PMSA were found to be quantitatively detected in urine of actual clinical patients by using a sensor according to an embodiment. As a result, it was found that diagnosis of diseases including prostate cancer is possible by a non-invasive urine test using a sensor according to an embodiment.

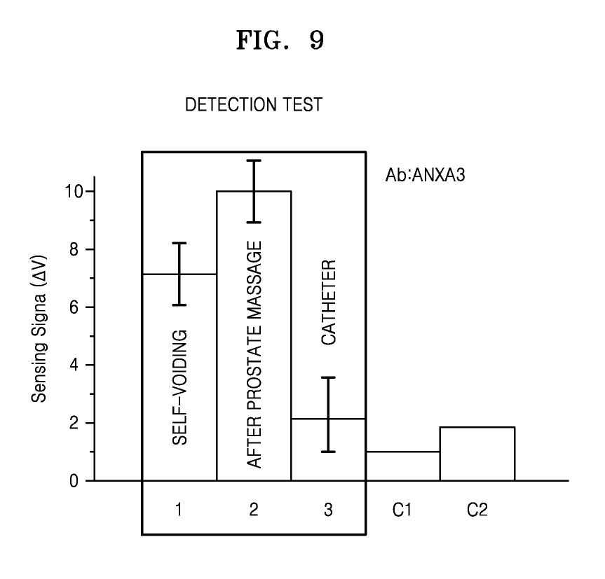

In general, even urine of the same patient may have different amounts of an analyte in a sample depending on a method of urine collection; in some cases, a marker may not even be detected depending on a method of urine collection. In order to confirm the effectiveness of urinalysis using a sensor according to an embodiment, irrespective of a method of urine sample collection, urine samples from each patient were collected through pre-operative self-voiding, through prostate massage, and through catheter during surgery, and then tested. Quantitative detection of ANX A3 in urine was performed, and urine samples from two healthy adults were used as a control group. The measurement results are shown in FIG. 9.

FIG. 9 is a histogram illustrating the results of measurement of ANX A3 by using a sensor according to an embodiment.

As shown in FIG. 9, the sensor according to an embodiment, irrespective of a method of urine sample collection, was found to accurately detect an amount of an analyte in the urine sample. Therefore, a sensor according to an embodiment may be used not only for early diagnosis of diseases such as prostate cancer, but also for monitoring the prognosis of a patient.

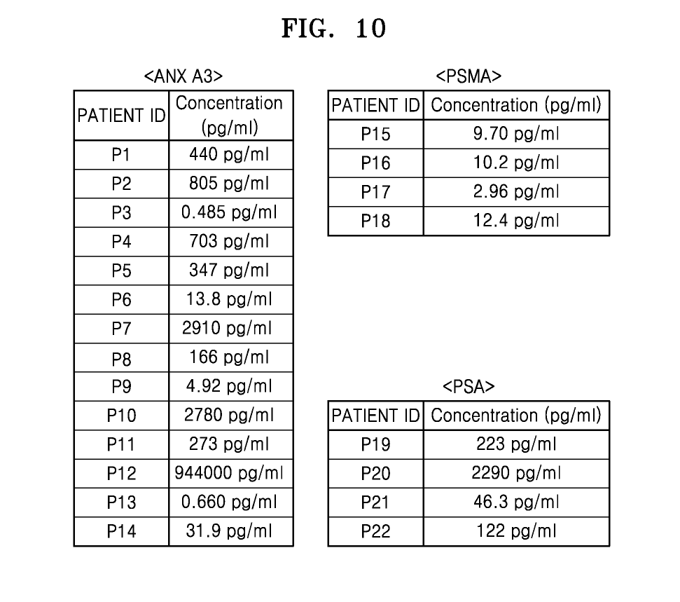

Next, the amounts of prostate cancer markers PSA, PSMA, and ANX A3 in urine samples of 22 prostate cancer patients were quantitatively detected in the same manner as described above. The measurement results are shown in FIG. 10.

FIG. 10 shows tables illustrating the results of measurements of PSA, ANX A3, and PSMA in patients' urine, the measurements obtained by using a sensor according to an embodiment.

As shown in FIG. 10, the amount of an analyte in urine samples were quantitatively measured using a sensor according to an embodiment. As a result, it can be seen that urinalysis may be performed accurately and easily by using a sensor according to an embodiment.

As apparent from the foregoing description, when a sensor according to an aspect is used to analyze urine for diagnosis and testing of a disease, it is possible to minimize patient's stress, clinical stress, and work burden, to accurately diagnose the patient in a comfortable condition, and to use the sensor for point-of-care (POC) diagnosis.

It should be understood that embodiments described herein should be considered in a descriptive sense only and not for purposes of limitation. Descriptions of features or aspects within each embodiment should typically be considered as available for other similar features or aspects in other embodiments.

While one or more embodiments have been described with reference to the figures, it will be understood by those of ordinary skill in the art that various changes in form and details may be made therein without departing from the spirit and scope of the disclosure as defined by the following claims.

What is claimed is:

1. A sensor for urinalysis, the sensor comprising:
an electrochemical sensing unit for detecting an analyte in urine and a signal processor for amplifying signals generated from the sensing unit, the signal processor comprising an ion-sensitive field-effect transistor (ISFET) electrically connected to the sensing unit,
wherein the sensing unit is separable from the signal processor,
the ISFET comprises a lower gate electrode; a lower insulating layer on the lower gate electrode; a source and a drain on the lower insulating layer and separated from each other; a channel layer on the lower insulating layer and between the source and the drain; an upper insulating layer on the source, the drain, and the channel layer; and an upper gate electrode on the upper insulating layer, and
an electrode of the sensing unit is electrically connected to the upper gate electrode of the ISFET.

2. The sensor of claim 1 further comprising a connecting portion for connecting the sensing unit to the signal processor.

3. The sensor of claim 1 further comprising a display unit for displaying results.

4. The sensor of claim 1, wherein the sensing unit comprises:
a substrate;
a working electrode and a reference electrode both on the substrate;
immobilized analyte binding materials on the working electrode; and
a test cell for accommodating the electrodes, the analyte binding materials, and an analyte.

5. The sensor of claim 4, wherein the sensing unit comprises a probe coupled to the analyte binding materials via the analyte in a sample and having a negative charge or a positive charge, wherein signals of the analyte are amplified by capacitive coupling of the probe to electrons in the channel layer of the ISFET.

6. The sensor of claim 4, wherein the analyte binding materials comprise deoxyribonucleic acids (DNA), ribonucleic acids (RNA), nucleotides, nucleosides, proteins, polypeptides, peptides, amino acids, carbohydrates, enzymes, antibodies, antigens, receptors, substrates, ligands, membranes, or a combination thereof.

7. The sensor of claim 4, wherein the analyte binding materials are antibodies that specifically bind to prostate-specific antigen (PSA), Annexin A3, or prostate-specific membrane antigen (PSMA), which are prostate cancer markers.

8. The sensor of claim 5, wherein the probe comprises metal nanoparticles.

9. The sensor of claim 1, wherein a thickness of an equivalent oxide layer of the upper insulating layer is smaller than a thickness of an equivalent oxide layer of the lower insulating layer.

10. The sensor of claim 1, wherein a thickness of the channel layer is 10 nanometers (nm) or less.

11. The sensor of claim 1, wherein the channel layer comprises any one selected from the group consisting of an oxide semiconductor, an organic semiconductor, polycrystalline silicon, and monocrystalline silicon.

12. The sensor of claim 1, wherein the sensor comprises a plurality of the sensing units and a plurality of the ISFETs, wherein the plurality of the sensing units are electrically connected to the plurality of the ISFETs, respectively.

13. The sensor of claim 12, wherein, in the plurality of the ISFETs, a plurality of sources are commonly grounded, a plurality of upper gate electrodes are commonly grounded, and a common voltage is applied to a plurality of lower gate electrodes.

14. The sensor of claim 12, wherein each of the plurality of sensing units independently comprises different immobilized analyte binding materials.

15. The sensor of claim 1, wherein the signal processor further comprises a calculation module electrically connected to the ISFET, the calculation module determining an amount of the analyte in urine from a potential difference measured by the ISFET.

16. The sensor of claim 15, wherein the calculation module determines an amount of a prostate cancer marker in the urine according to a graph shown in FIG. 7A, 7B, or 7C.
